(19) United States
(12) Patent Application Publication
(10) Pub. No.: US 2004/0141927 A1
(43) Pub. Date: Jul. 22, 2004

(54) Oral products containing novel flavor composition

(76) Inventors: Sonya S. Johnson, LaGrange, IL (US); Gloria Sheldon, Hammond, IN (US); Michael J. Greenberg, Northbrook, IL (US)

Correspondence Address: BELL, BOYD & LLOYD LLC, P. O. BOX 1135, CHICAGO, IL, 60690-1135, US

(21) Appl. No.: 10/705,481

(22) Filed: Nov. 10, 2003

Related U.S. Application Data
(60) Provisional application No. 60/319,694, filed on Nov. 14, 2002.

(51) Int. Cl. A61K 9/68; A61K 7/26; A61K 35/78

(52) U.S. Cl. 424/48; 424/776; 424/58

(57) ABSTRACT

Oral products containing Erospicata oil and a cooling agent for a novel organoleptic experience and cost savings. The oral products of this invention include chewing gums, confections, mouthwashes, mouth sprays and edible thin film products.

### BACKGROUND OF THE INVENTION

There is considerable consumer demand for novelty in the experience of oral products and confections. Products are continually developed with new ingredients and combinations of ingredients to provide unique oral sensations and flavors through the consumption of an oral product for the enjoyment and satisfaction of the consumer.

Of course, flavor and taste factor heavily into marketability of an oral product.

In addition, manufacturers are continually looking to reduce production costs. Manufacturers inquire into manufacturing processes and also ingredients used in oral products and provides greater value for the consumer.

### SUMMARY OF THE INVENTION

The present invention relates to methods of enhancing flavor in oral products. Furthermore, the present invention relates to reducing the costs associated with producing an oral product. Specifically, the present invention relates to oral products containing Erospicata oil with a cooling agent. More specifically, the present invention relates to a dentifrice, toothpaste, oral creams and lotions, chewing gum, confection, lozenge, mouthwash, mouth spray or edible film containing Erospicata oil and a cooling agent which provides a novel flavor. Cooling agents, for the purpose of the present invention are defined as non-menthol cooling agents.

More specifically, the present invention relates to a dentifrice, chewing gum, confection, lozenge, mouthwash, mouth spray or edible film containing Erospicata oil and a cooling agent which reduces cost of peppermint flavored products by way of blending Erospicata oil and cooling agents. Optionally, the present invention may also contain a heating agent to potentiate the cooling agent. Optionally, the cooling agent may be substituted with a heating agent for a desired effect of heat, tingle or itch. Other consumer products which may incorporate this invention are perfumes, after shaves, shampoos, topical creams and lotions and the like.

In an embodiment of the present invention, the oral product is chewing gum or any variation, including but not limited to, bubble gums, pellets, gum balls, tabs or sticks. Chewing gums may be coated or not coated and be of a variety of flavors, shapes and sizes.

In an embodiment of this invention, the oral product is a confectionery composition including but not limited to hard candy, chewing candy, filled candy and pressed tablets.

In another embodiment of the present invention, the oral product is a thin edible film.

In another embodiment the oral product is a dentifrice.

Additional features and advantages of the present invention are described in, and will be apparent from, the following Detailed Description of the Invention and the figures.

### DETAILED DESCRIPTION OF THE INVENTION

The present invention incorporates Erospicata oil as one of the ingredients of an oral product. Erospicata oil is disclosed in U.S. Patent Plant 8,645, herein incorporated by reference. Erospicata is a novel Mentha spicata mint plant which is resistant to verticillium wilt and mint rust. The Mentha spicata also has a low 1-menthol content and is high in menthone. The oil provided by the Mentha spicata has a taste similar to de-mentholized peppermint and has a hot odor. Because this plant is resistant to verticillium wilt and mint rust, the viability of these plants is much higher than non-resistant strains, which allows for lower cost of the end product of oil. The Erospicata oil is available for purchase from RCB International, Ltd., located at 39878 Turnridge Road, N.E., Albany, Oreg., USA. RCB has offered for sale certain blends of Erospicata oil, peppermint oil and menthol.

In addition to the oral products containing Erospicata oil, the oral products of the present invention also contain flavors called cooling agents or high intensity cooling agents. Cooling agents enhance the flavor and perceived breath freshening of the product. Cooling agents include N-ethyl-p-menthane-3-carboxamide (WS-3), N,2,3-trimethyl-2-isopropyl-butanamide (WS-23), menthyl glutarate, menthyl succinate, menthol PG carbonate, menthol EG carbonate, menthyl lactate, menthone glyceryl ketal, menthol glyceryl ether, 3,3,5-trimethylcyclohexanol (Homomenthol), isopulegol and combinations thereof.

The present invention may also optionally contain a heating agent. The heating agent, in small amounts, potentiates the effect of the cooling agent. The heating agent may also substitute for the cooling agent in the present invention if the experience of heat, tingling or itching is desired. The heating agents of the present invention are capsicum oleoresin, capsaicin, piperine, gingerol, shoagol, cinnamic aldehyde, ginger oleoresin, cinnamon and cassia oleoresin, black pepper oleoresin, pepper oleoresin and combinations thereof. In the present invention, generally, the heating agent may be used in amounts of about 0.01% to about 2%. Specifically, capsicum oleoresin, capsaicin, piperine, gingerol and shoagol could be used in quantities of about 0.01%-0.03%, cinnamic aldehyde could be used in amount of 1%-2%, the oleoresins could be used from about 0.05% or less.

Flavoring agents of Erospicata oil and additional cooling agents in the present invention are present in amounts of 0.05-15% by weight of oral product. Preferably the flavoring agents of the present invention are present in amounts of 0.2-5.0% by weight of oral product. The composition of the flavoring agent may be 5%-70% Erospicata by weight and 5%-80% cooling agent by weight, and any remaining percentage by other flavors known in the art.

In an embodiment, the oral composition comprises an effective amount of Erospicata oil and an effective amount of cooling agent selected from the group consisting of N-ethyl-p-menthane-3-carboxamide, N,2,3-trimethyl-2-isopropyl-butanamide, menthyl glutarate, menthyl succinate, menthol PG carbonate, menthol EG carbonate, menthyl lactate, menthone glyceryl ketal, menthol glyceryl ether, 3,3,5-trimethylcyclohexanol, isopulegol, and combinations thereof.

In an embodiment, the oral composition comprises an effective amount of Erospicata oil and an effective amount of menthyl glutarate.

In an embodiment, the oral composition comprises an effective amount of Erospicata oil and an effective amount of N,2,3-trimethyl-2-isopropyl-butanamide.

In an embodiment, the oral composition comprises an effective amount of Erospicata oil and an effective amount of menthol glyceryl ether.

In an embodiment, the oral composition additionally comprises an effective amount of menthol.

In an embodiment, a chewing gum comprising Erospicata oil and cooling agent selected from the group consisting of menthol, menthol glyceryl ether, N,2,3-trimethyl-2-isopropyl-butanamide and menthyl glutarate or combinations thereof.

There are several methods which may be used to enhance the release of the Erospicata oil and other flavoring agents, such as cooling agents, from the oral composition. In a chewing gum product, the gum base is hydrophilic which would facilitate the release of the Erospicata oil and other flavors. In an oral composition, the Erospicata oil and other flavoring agents may be encapsulated, spray dried, formulated into the coating and combinations thereof.

In an embodiment of the present invention, Erospicata oil and a cooling agent is present in a chewing gum formulation. In an embodiment of the present invention, the amount of Erospicata oil present is up to about 5% by weight of the chewing gum product, and cooling agent is present up to about 5% by weight of the chewing gum product. In an embodiment of the present invention, the amount of Erospicata oil is about 1% of the weight of the chewing gum product and the cooling agent is present at about 1% by weight of the chewing gum product. In another embodiment, the Erospicata oil is present in the amount of about 0.25% by weight of the chewing gum product and the cooling agent is present at about 1% by weight of the chewing gum product. In another embodiment, the Erospicata oil is present in the amount of about 0.01% by weight of the chewing gum product and the cooling agent is present at about 0.05% by weight of the chewing gum product.

In general, a chewing gum composition typically comprises a water-soluble bulk portion, a water-insoluble chewable gum base portion and typically water-insoluble flavoring agents. The water-soluble portion dissipates with a portion of the flavoring agent over a period of time during chewing. The gum base portion is retained in the mouth throughout the chew.

The insoluble gum base generally comprises elastomers, resins, fats and oils, softeners and inorganic fillers. The gum base may or may not include wax. The insoluble gum base can constitute approximately 5% to about 95% by weight of the chewing gum, more commonly the gum base comprises 10% to about 50% of the gum, and in some preferred embodiments approximately 25% to about 35% by weight, of the chewing gum.

In a particular embodiment, the chewing gum base of the present invention contains about 20% to about 60% by weight synthetic elastomer, up to about 30% by weight natural elastomer, about 5% to about 55% by weight elastomer plasticizer, about 4% to about 35% by weight filler, about 5% to about 35% by weight softener, and optional minor amounts (about 1% or less by weight) of miscellaneous ingredients such as colorants, antioxidants, etc.

Synthetic elastomers may include, but are not limited to, polyisobutylene with GPC weight average molecular weight of about 10,000 to about 95,000, isobutylene-isoprene copolymer (butyl elastomer), styrenecopolymers having styrene-butadiene ratios of about 1:3 to about 3:1, polyvinyl acetate having GPC weight average molecular weight of about 2,000 to about 90,000, polyisoprene, polyethylene, vinyl acetate vinyl laurate copolymer having vinyl laurate content of about 5% to about 50% by weight of the copolymer, and combinations thereof.

Preferred ranges for polyisobutylene are 50,000 to 80,000 GPC weight average molecular weight and for styreneare 1:1 to 1:3 bound styrene for polyvinyl acetate are 10,000 to 65,000 GBC weight average molecular weight with the higher molecular weight polyvinyl acetates typically used in bubble gum base, and for vinyl acetatelaurate, vinyl laurate content of 10.

Natural elastomers may include natural rubber such as smoked or liquid latex and guayule as well as natural gums such as jelutong, lechi caspi, perillo, sorva, massaranduba balata, massaranduba chocolate, nispero, rosindinha, chicle, gutta hang kang, and combinations thereof. The preferred synthetic elastomer and natural elastomer concentrations vary depending on whether the chewing gum in which the base is used is adhesive or conventional, bubble gum or regular gum, as discussed below. Preferred natural elastomers include jelutong, chicle, sorva and massaranduba balata.

Elastomer plasticizers may include, but are not limited to, natural rosin esters such as glycerol esters or partially hydrogenated rosin, glycerol esters of polymerized rosin, glycerol esters of partially dimerized rosin, glycerol esters of rosin, pentaerythritol esters of partially hydrogenated rosin, methyl and partially hydrogenated methyl esters of rosin, pentaerythritol esters of rosin; synthetics such as terpene resins derived from alpha beta and/or any suitable combinations of the foregoing. The preferred elastomer plasticizers will also vary depending on the specific application, and on the type of elastomer which is used.

Fillers/texturizers may include magnesium and calcium carbonate, ground limestone, silicate types such as magnesium and aluminum silicate, clay, alumina, talc, titanium oxide, mono-, di- and tri-phosphate, cellulose polymers, such as wood, and combinations thereof.

Softeners/emulsifiers may include tallow, hydrogenated tallow, hydrogenated and partially hydrogenated vegetable oils, cocoa butter, glycerol monostearate, glycerol triacetate, lecithin, mono and triglycerides, acetylated monoglycerides, fatty acids (e.g. stearic, palmitic, oleic and linoleic acids), and combinations thereof.

Colorants and whiteners may include FD&C dyes and lakes, fruit and vegetable extracts, titanium dioxide, and combinations thereof.

The base may or may not include wax. An example of a wax gum base is disclosed in U.S. Pat. No. 5,286,500, the disclosure of which is incorporated herein by reference.

In addition to a water insoluble gum base portion, a typical chewing gum composition includes a water soluble bulk portion and one or more flavoring agents. The water soluble portion can include bulk sweeteners, high intensity sweeteners, flavoring agents, softeners, emulsifiers, colors, acidulants, fillers, antioxidants, and other components that provide desired attributes.

Softeners are added to the chewing gum in order to optimize the chewability and mouthfeel of the gum. The softeners, which are also known as plasticizers and plasticizing agents, generally constitute between approximately 0.5% to about 15% by weight of the chewing gum. The softeners may include glycerin, lecithin, and combinations thereof. Aqueous sweetener solutions such as those containing sorbitol, hydrogenated starch hydrolysates, corn syrup and combinations thereof, may also be used as softeners and binding agents in chewing gum.

Bulk sweeteners include both sugar and sugarless components. Bulk sweeteners typically constitute about 5% to about 95% by weight of the chewing gum, more typically, about 20% to about 80% by weight, and more commonly, about 30% to about 60% by weight of the gum. Sugar sweeteners generally include saccharide components commonly known in the chewing gum art, including but not limited to, sucrose, dextrose, maltose, dextrin, dried invert sugar, fructose, levulose, galactose, corn syrup solids, and the like, alone or in combination. Sugarless sweeteners include, but are not limited to, sugar alcohols such as sorbitol, mannitol, xylitol, hydrogenated starch hydrolysates, maltitol, and the like, alone or in combination.

High intensity artificial sweeteners can also be used, alone or in combination, with the above. Preferred sweeteners include, but are not limited to, sucralose, aspartame, NAPM derivatives such as neotame, salts of acesulfame, altitame, saccharin and its salts, cyclamic acid and its salts, glycyrrhizinate, dihydrochalcones, thaumatin, monellin, and the like, alone or in combination. In order to provide longer lasting sweetness and flavor perception, it may be desirable to encapsulate or otherwise control the release of at least a portion of the artificial sweetener. Such techniques as wet granulation, wax granulation, spray drying, spray chilling, fluid bed coating, coacervation, and fiber extension may be used to achieve the desired release characteristics.

Combinations of sugar and/or sugarless sweeteners may be used in chewing gum. Additionally, the softener may also provide additional sweetness such as with aqueous sugar or alditol solutions.

If a low calorie gum is desired, a low caloric bulking agent can be used. Examples of low caloric bulking agents include: polydextrose; Raftilose, Raftilin; Fructooligosaccharides (NutraFlora); Palatinose oligosaccharide; Guar Gum Hydrolysate (Sun Fiber); or indigestible dextrin (Fibersol). However, other low calorie bulking agents can be used.

A variety of flavoring agents can also be used, if desired. The flavor can be used in amounts of about 0.1 to about 15 weight percent of the gum, and preferably, about 0.2% to about 5% by weight. Flavoring agents may include essential oils, synthetic flavors or mixtures thereof including, but not limited to, oils derived from plants and fruits such as citrus oils, fruit essences, peppermint oil, spearmint oil, other mint oils, clove oil, oil of wintergreen, anise and the like. Artificial flavoring agents and components may also be used. Natural and artificial flavoring agents may be combined in any sensorially acceptable fashion.

In addition, to the active ingredients of the present invention, additional active ingredients or medicaments may be added for various purposes. If the medicament or active is water soluble in the chewing gum, it preferably will include a base/emulsifier system which leads to the desired concentration of the medicament in the saliva (more hydrophilic balance). If the medicament or active is water insoluble, the chewing gum preferably includes a base/emulsifier system which leads to the desired concentration of the medicament in the saliva (more lipophilic balance).

In manufacturing the chewing gum including the active agent or ingredient, the active agent or medicament is added, preferably, early on in the mix. The smaller the amount of active ingredient used, the more necessary it becomes to preblend that particular ingredient to assume uniform distribution throughout the batch of gum. Whether a preblend is used or not, the active agent or medicament should be added within the first five minutes of mixing. For faster release, the active agent may be added late in the process.

Optionally, the chewing gum of the present invention may include additional breath freshening, anti-microbial or oral health ingredients. Food acceptable metallic salts selected from zinc and copper salts of gluconic acid, zinc and copper salts of lactic acid, zinc and copper salts of acetic acid, zinc and copper salts of citric acid and combinations thereof.

Anti-microbial essential oils and flavor components such as peppermint, methyl salicylate, thymol, eucalyptol, cinnamic aldehyde, polyphosphate, pyrophosphate and combinations thereof.

Dental health ingredients such as fluoride salts, phosphate salts, proteolytic enzymes, lipids, anti-microbials, calcium, electrolytes, protein additives, dental abrasives and combinations thereof.

Optionally, chewing gum may also be coated. Techniques for coating chewing gum may be by methods such as syrup panning, dry charge and the like. The chewing gum coating may contain active ingredients such as colors, flavors, acids, high intensity sweeteners, anti-microbial agents such as metallic salts and essential oils, medicaments, dental health ingredients, cooling agents, heating agents and combinations thereof. These active agents may be spray dried or encapsulated in the coating.

In general, chewing gum is manufactured by sequentially adding the various chewing gum ingredients to a commercially available mixer known in the art. After the ingredients have been thoroughly mixed, the gum mass is discharged from the mixer and shaped into the desired form such as rolling sheets and cutting into sticks, extruding into chunks or casting into pellets, which are then coated or panned. The gum mass may be shaped into balls, tabs, sticks, pellets or variations thereof. The gum mass may also be processed by coextrusion and also shaped by nipping techniques.

Generally, the ingredients are mixed by first melting the gum base and adding it to the running mixer. The base may also be melted in the mixer itself. Color or emulsifiers may also be added at this time. A softener such as glycerin may also be added at this time, along with syrup and a portion of the bulking agent. Further parts of the bulking agent are added to the mixer. Flavoring agents are typically added with the final portion of the bulking agent. Other optional ingredients are added to the batch in a typical fashion, well known to those of ordinary skill in the art.

The entire mixing procedure typically takes from five to fifteen minutes, but longer mixing times may sometimes be required. Those skilled in the art will recognize that many variations of the above described procedure may be followed. After mixing, the chewing gum is shaped and wrapped. The chewing gum may be of a variety of shapes and sized including sticks, tabs, pellets, balls or any variations thereof.

Chewing gum base and chewing gum product have been manufactured conventionally using separate mixers, different mixing technologies and, often, at different factories. One reason for this is that the optimum conditions for manufacturing gum base, and for manufacturing chewing gum from gum base and other ingredients such as sweeteners and flavors, are so different that it has been impractical to integrate both tasks. Chewing gum base manufacture, on the one hand, involves the dispersive (often high shear) mixing of difficult-to-blend ingredients such as elastomer, filler, elastomer plasticizer, base softeners/emulsifiers and sometimes wax, and typically requires long mixing times. Chewing gum product manufacture, on the other hand, involves combining the gum base with more delicate ingredients such as product softeners, bulk sweeteners, high intensity sweeteners and flavoring agents using distributive (generally lower shear) mixing, for shorter periods.

Chewing gum may also be manufactured continuously, utilizing mixing extruders or other suitable equipment. One such method is described in U.S. Pat. No. 5,543,160 entitled Total Chewing Gum Manufacture Using High Efficiency Continuous Mixing, which is herein incorporated by reference. In addition, U.S. Pat. No. 5,800,847, entitled Total Chewing Gum Manufacture Using High Efficiency Continuous Mixing, which is herein incorporated by reference, also describes this process. The method uses a continuous high efficiency mixer which is configured for the total manufacture of a wide variety of chewing gum products. By employing continuous mixing and/or integrating the manufacture of chewing gum base and chewing gum into a single operation, it is possible to reduce time and labor costs and improve product consistency.

The following are examples of formulations of Erospicata oil in chewing gum. The examples are not intended to exclude other variations in formulations and the present invention is not limited to these formulations.

TABLE 1
Novel Flavor Gum Formulas (% by weight)

| Ingredient | Example 1 | Example 2 | Example 3 | Example 4 | Example 5 | Example 6 |
|---|---|---|---|---|---|---|
| Gum Base | 26.15 | 26.05 | 26.00 | 27.5 | 27.5 | 28.50 |
| Talc powder | 3.00 | 3.00 | 3.00 | 3.00 | 3.00 | 2.50 |
| Glycerine | 0.50 | 0.50 | 0.50 | 0.50 | 0.50 | 0.45 |
| Sorbitol | 49.71 | 50.96 | 48.86 | 48.86 | 44.86 | 47.75 |
| Glycerol | 15.01 | 15.11 | 15.01 | 15.01 | 15.01 | 14.12 |
| Mannitol | 1.52 | 1.52 | 1.52 | 1.52 | 1.52 | 1.51 |
| Maltitol | 0.76 | 0.76 | 0.76 | 0.76 | 0.76 | 0.74 |
| Water | 1.18 | 1.18 | 1.18 | 1.18 | 1.18 | 1.19 |
| Aspartame | 0.53 | 0.53 | 0.53 | 0.53 | 0.53 | 0.50 |
| Color | 0.01 | 0.01 | 0.01 | 0.01 | 0.01 | 0.02 |
| Acesulfame-K | 0.13 | 0.13 | 0.13 | 0.13 | 0.13 | 0.12 |
| Erospicata | 0.08 | 0.13 | 0.33 | 0.77 | 3.10 | 1.82 |
| Cooling Agent | 1.12 | 0.11 | 0.90 | 0.15 | 0.95 | 0.26 |
| Peppermint | 0.30 | 0.01 | 1.27 | 0.08 | 0.95 | 0.52 |
| Total % | 100.00 | 100.00 | 100.00 | 100.00 | 100.00 | 100.00 |

TABLE 2
Novel Flavor Gum Formulas (% by weight)

| Ingredient | Example 7 | Example 8 | Example 9 | Example 10 | Example 11 | Example 12 |
|---|---|---|---|---|---|---|
| Gum Base | 19.46 | 20.71 | 19.46 | 19.46 | 18.46 | 19.65 |
| Sugar | 62.13 | 62.13 | 61.13 | 62.63 | 61.63 | 62.69 |
| Corn Syrup | 15.57 | 15.57 | 15.57 | 15.57 | 13.57 | 15.57 |
| Color | 0.67 | 0.67 | 0.67 | 0.67 | 0.67 | 0.67 |
| Glycerin | 0.67 | 0.67 | 0.67 | 0.67 | 0.67 | 0.67 |
| Erospicata | 0.23 | 0.09 | 1.12 | 0.70 | 1.00 | 0.25 |
| Cooling Agent | 0.29 | 0.07 | 1.23 | — | 1.00 | 0.25 |
| Heating Agent | 0.01 | — | — | 0.10 | — | — |
| Peppermint | 0.97 | 0.07 | 0.15 | 0.18 | 3.00 | 0.25 |
| Menthol | — | 0.02 | — | 0.02 | — | — |
| Total % | 100.00 | 100.00 | 100.00 | 100.00 | 100.00 | 100.00 |

In an embodiment of the present invention, and effective amount for unique flavoring benefit, Erospicata oil and cooling agent are present in an edible film formulation. In an embodiment of the present invention, the amount of Erospicata oil present is up to 20% by weight of the edible film formulation. In an embodiment of the present invention, the amount of Erospicata oil is about 8% by of the weight of the edible film product and the cooling agent is about 8% by weight of the edible film product. In another embodiment, the Erospicata oil is present in the amount of about 5% by weight of the edible film product and the cooling agent is about 10% by weight of the edible film product. In an embodiment, the amount of Erospicata oil present is in an amount above 15% and the cooling agent is about 5% by weight of the edible film product. In an embodiment, the amount of Erospicata oil is present is in amount above 5% and the cooling agent is about 10% by weight of the edible film product. In an embodiment, the amount of Erospicata oil present in an amount between 6% and 25% and the cooling agent is between 5% and 15% by weight of the edible film product.

The present invention provides edible film formulations for oral mucoadhesion and methods of using and making same. In particular, the edible films of the present invention include at least four types of film forming agents.

In the present invention film forming agents may be maltodextrins, fillers (e.g., microcrystalline cellulose (MCC)) and hydrocolloids (e.g., sodium aliginate), pullulan and combinations thereof. These film forming agents, separately or in combination, can be effectively utilized to prepare stand alone edible films. The edible films are composed of ingredients that are readily available. Pullulan free edible films can be prepared at lower costs and display similar properties as compared to edible films composed of pullulan. Although, for the present invention, pullulan may also be used as an ingredient in the edible film formulations. In this regard, the edible films can provide a physiologically acceptable film, which is suitably adapted to adhere to oral surfaces of an oral cavity and rapidly dissolve therein.

The edible films of the present invention can be utilized to deliver or release oral care agent(s). Such agents include, anti-microbial agents and salivary stimulants to treat, for example, halitosis, dental plaque, gingivitis, xerostomia, dry mouth, like oral conditions or combinations thereof. Further, the oral care edible film can act as a breath freshener effective against malodor.

The oral cleansing and breath freshening effects of the edible film of the present invention can be achieved by entrapping the oral care agents within the oral cavity to provide extended efficacy. In this regard, the highly dissolvable edible film can act as a medium through which a pharmaceutically active oral agent can be administered via a mucous membrane of the oral cavity.

Further, the edible films can include a variety of other suitable ingredients, such as softeners, colorants, flavoring agents, emulsifiers, surfactants, thickening agents, binding agents, sweeteners, fragrances, other like ingredients or combinations thereof.

In an embodiment, the edible films preferably include a mixture of at least three types of film forming agents, such as maltodextrins, fillers and hydrocolloids. It should be appreciated that the edible film of the present invention can be composed of one or more different compounds associated with each of the at least three types of film forming agents.

In an embodiment, the maltodextrin component constitutes between about 5% to about 60% by dry weight of the edible film, preferably about 20% to about 40% by dry weight. The maltodextrin component can be processed in any suitable way.

The hydrocolloid can provide thickness and decrease brittleness of the edible films. The hydrocolloid can include any suitable type, amount and number of hydrocolloids. In an embodiment, the hydrocolloid can constitute between about 10% to about 50% by dry weight of the edible film, preferably about 20% to about 30% by dry weight. The hydrocolloid can be derived from, for example, natural seaweeds, natural seed gum, natural plant exudates, natural fiber extracts, biosynthetic gums, gelatins, biosynthetic process starch or cellulosic materials, alginates, sodium alginate, calcium alginate, carrageenans, guar gum, locust gum, tara gum, gum arabic, ghatti gum, agar gum, xanthan gum, pectin, other like hydrocolloid source material or combinations thereof.

Any suitable food-grade bulk filler can also be added to the edible film. This can reduce any slimy texture as well as provide structure to the film thereby making it more palatable. In an embodiment, the filler can constitute about 5% to about 30% by dry weight of the film, preferably about 15% to about 25% by dry weight. The filler can include, for example, microcrystalline cellulose, cellulose polymers, such as wood, magnesium and calcium carbonate, ground limestone, silicates, such as magnesium and aluminum silicate, clay, talc, titanium dioxide, mono-calcium phosphate, di-calcium phosphate, tri-calcium phosphate, other like bulk fillers or combinations thereof.

As previously discussed, a variety of other suitable ingredients can be added to the edible film of the present invention. For example, any suitable medicament for oral cleansing, breath freshening or the like can be added to the film formulation. The medicaments can include, for example, a pH control agent, such as urea and buffers, inorganic components for tartar or caries control, such as phosphates and fluorides, a breath freshening agent such as zinc gluconate, an anti-plaque/anti-gingivitis agent, such as cholorhexidene, CPC, and triclosan, a saliva stimulating agent including, for example, food acids such as citric, lactic, maleic, succinic, ascorbic, adipic, fumaric and tartaric acids, a pharmaceutical agent, a nutraceutical agent, a vitamin, a mineral, other like medicaments or combinations thereof.

The medicaments can be delivered or released into the oral cavity for effective oral treatment, such as oral cleansing and/or breath freshening. In this regard, the film forming agent of the edible film can act to entrap the medicaments within the oral cavity thereby providing extended efficacy thereof. Moreover, it is also believed that the mixture of film-forming agents of the present invention can entrap the medicament within the oral cavity for an extended period of time to prolong and enhance the effects of the medicament. In addition, by extending the contact time of the medicament within the oral cavity, the medicament is absorbed to a greater extent thereby increasing its bioavailability.

If reduced levels of film forming agents are utilized, softeners can be used to reduce the brittleness of the resulting films. The softeners, which are also known as plasticizers or plasticizing agents, generally constitute between about up to 20% by dry weight of the film, preferably about 2% to about 10% by dry weight. The softeners can include plasticizers containing, for example, sorbitol and other polyols, glycerin, polyethylene glycol, propylene glycol, hydrogenated starch hydrolysates, corn syrups, other like material or combinations thereof.

The edible film formulations of the present invention can also include colorants or coloring agents which can be used in any suitable amount to produce the desired color. Coloring agents can include, for example, natural food colors and dyes suitable for food, drug and cosmetic applications. The colorants are typically knows as FD&C dyes and lakes.

A variety of flavoring agents can also be added to the edible films. Any suitable amount and type of artificial and/or natural flavoring agents can be used in any sensorially acceptable fashion. For example, the flavor can constitute about 0.1% to about 20% by dry weight of the film, preferably about 10% to 15%. The flavoring agent can include, for example, essential oils, synthetic flavors or mixtures including but not limited to oils delivered from plants and fruits such as citrus oils, fruit essences, peppermint oil, spearmint oil, other mint oils, clove oils, oil of wintergreen, anise and the like, flavor oils with germ killing properties such as menthol, eucalyptol, thymol, like flavoring agents or combinations thereof.

The flavor can be enhanced and evenly distributed throughout the product by emulsification. Any suitable amount and type of natural and/or synthetic food-grade emulsifier can be used. For example, the emulsifier can include lecithin, food-grade non-ionic emulsifiers, such as fatty acids (C₁₀-C₁₈), mono and diacyl glycerides, ox bile extract, polyglycerol esters, polyethylene sorbitan esters, propolyene glycol, sorbitan monopalmitate, sorbitan monosterate, sorbitan tristerate, enzyme modified lecithin, hyroxylated lecithins, other like emulsifiers or combinations thereof.

The flavors can be emulsified by any suitable emulsification process, such as mechanical processing, vigorous stirring, intense pressure fluctuations that occur in turbulent flow such as homogenization, sonication, colloid milling and the like.

The present invention provides methods of producing the edible film formulations. In general, the edible film formulations are prepared by forming a base solution that includes at least three types of film forming agents, such as maltodextrins, hydrocolloids and fillers and processing the base solution to form an edible film. Typically, the base solution is prepared by adding an initial mixture of dry ingredients to water that is stirred.

To the base solution, additional ingredients, such as flavor/emulsifier blends, sweeteners, softeners, color, the like or combinations thereof, can be added. In an embodiment, the solution is stirred continuously and heated at a temperature ranging from about 40° C. to about 60° C. The solution then can be dried in any suitable manner, thereby, forming the edible film.

It should be appreciated that any suitable type, number and arrangement of process procedures or steps (i.e. mixing, heating, drying, cooling, addition of ingredients), process parameters (i.e. temperature, pressure, pH, process times) or the like can be utilized.

By way of example and not limitation, the following examples illustrate various embodiments of the edible film formulations of the present invention.

TABLE 3
Novel Flavor Thin Film Formulas (% by weight and dry basis)

| Ingredient | Example 13 | Example 14 | Example 15 | Example 16 | Example 17 |
|---|---|---|---|---|---|
| Water | 11.00 | 10.00 | 10.00 | 10.00 | 10.00 |
| Maltodextrin | 26.00 | 23.23 | 24.56 | 25.96 | 23.00 |
| Sodium Alginate | 28.79 | 27.33 | 21.67 | 25.37 | 21.70 |
| Carageenan | 8.66 | 8.51 | 9.26 | 7.73 | 6.54 |
| Microcrystalline Cellulose | 8.75 | 7.02 | 9.12 | 9.56 | 6.58 |
| Hydroxylated Lecithin | 2.12 | 1.86 | 2.11 | 3.01 | 5.50 |
| Glycerin | 7.35 | 6.92 | 8.33 | 6.56 | 6.79 |
| Cooling Agent | 3.71 | 5.40 | 3.77 | 1.50 | 3.26 |
| Peppermint | 0.35 | 0.60 | 5.35 | 0.80 | 3.26 |
| Erospicata | 0.09 | 6.00 | 1.36 | 7.70 | 10.63 |
| Sucralose | 3.13 | 3.08 | 4.42 | — | — |
| High Intensity Sweetener | — | — | — | 1.76 | 1.98 |
| Color | 0.05 | 0.05 | 0.05 | 0.05 | 0.76 |
| Total % | 100.00 | 100.00 | 100.00 | 100.00 | 100.00 |

TABLE 4
Novel Flavor Edible Film Formulations (% by weight)

| Ingredient | Example 18 | Example 19 | Example 20 | Example 21 | Example 22 |
|---|---|---|---|---|---|
| Maltodextrin | 17.95 | — | 31.20 | 36.80 | 21.00 |
| Pullulan | 30.00 | 46.00 | — | — | — |
| Sodium Alginate | — | — | 19.00 | — | 12.00 |
| Calcium Alginate | — | 15.15 | — | 11.45 | — |
| Carageenan | — | — | — | — | 12.00 |
| Microcrystalline Cellulose | 25.75 | 9.00 | 18.80 | 13.50 | 20.00 |
| Calcium Carbonate | — | 2.45 | — | — | — |
| Glycerin | 12.75 | 11.05 | 8.00 | — | 9.5 |
| Sorbitol | — | — | — | 6.00 | 1.55 |
| Propylene Glycol | — | — | 3.65 | 5.75 | — |
| Cooling Agent | 7.28 | 2.80 | 5.95 | 10.78 | 2.00 |
| Eucalyptol | — | 0.05 | — | 1.00 | — |
| Maleic Acid | — | — | — | — | 1.35 |
| Citric Acid | — | — | 1.25 | — | 1.00 |
| Chlorohexidene | 1.85 | — | — | 1.00 | — |
| Triclosan | — | 1.25 | — | 1.00 | — |
| Peppermint | 2.08 | 9.10 | 5.10 | 1.32 | 4.00 |
| High Intensity Sweetener | 1.25 | 1.00 | 1.05 | 1.45 | 1.50 |
| Erospicata | 1.04 | 2.10 | 5.95 | 9.90 | 14.00 |
| Color | 0.05 | 0.05 | 0.05 | 0.05 | 0.10 |
| Total % | 100.00 | 100.00 | 100.00 | 100.00 | 100.00 |

TABLE 5
Novel Flavor Edible Film Formulations (% by weight)

| Ingredient | Example 23 | Example 24 | Example 25 | Example 26 | Example 27 |
|---|---|---|---|---|---|
| Maltodextrin | 35.00 | 30.35 | — | 25.00 | 30.00 |
| Sodium Alginate | 22.15 | 19.10 | — | 28.15 | — |
| Carageenan | — | — | 12.15 | — | 20.15 |
| Pullulan | | | 47.00 | | — |
| Microcrystalline Cellulose | 20.00 | 18.00 | — | 17.00 | 18.00 |
| Gum Arabic | — | — | 11.00 | — | — |
| Glycerin | 7.30 | 15.00 | 10.30 | 7.30 | 7.30 |
| Erospicata | 0.72 | 4.66 | 9.92 | 9.50 | 3.15 |
| Lecithin | 2.00 | 2.00 | 2.00 | 2.00 | 2.00 |
| High Intensity Sweetener | 1.50 | 1.50 | 1.50 | 1.50 | 1.50 |
| Coolin Agent | — | 3.67 | 3.03 | 8.55 | 4.20 |
| Heating Agent | 1.43 | — | 0.01 | — | — |
| Peppermint | 9.84 | 4.67 | 3.04 | 0.95 | 13.65 |
| Color | 0.05 | 0.05 | 0.05 | 0.05 | 0.05 |
| Total % | 100.00 | 100.00 | 100.00 | 100.00 | 100.00 |

In yet another embodiment of the present invention, and effective amount of Erospicata oil and cooling agent are present for novel flavor experience are present in a confectionery formulation. In an embodiment of the present invention, the amount of Erospicata oil is present in an amount up to 3% by weight of the confectionery product and the cooling agent is about 3% by weight of the confectionery product. In an embodiment of the present invention, the amount of Erospicata oil is about 1% of the weight of the confectionery product and the cooling agent is about 0.5% by weight of the confectionery product. In another embodiment, the Erospicata oil is present in the amount of about 0.01% by weight of the confectionery product and the cooling agent is about 1% by weight of the confectionery product.

Confectionery products for this invention may be hard candies, chewy candies, coated chewy center candies and tabletted candies. By way of example, the hard candy is primarily comprised of corn syrup and sugar, and derives its name from the fact that it contains only 1.0% and 4% moisture. In appearance, these types of candies are solid, but they are actually supercooled liquids, which are far below their melting points. There are different types of hard candies. Glass types are usually clear or made opaque with dyes; and Grained Types, which are always opaque.

The continuous making process of the Deposited Glass Types, with a sugar base are as follows. Sugar corn syrup mixture is spread over a cylinder heated by high pressure steam. Rapid head exchange causes the water in the syrup to evaporate. The cooked syrup is discharged, colors and flavors are added. These can be conveyed directly to hoppers which then discharge directly into molds.

The candy is conveyed to batch rollers, which shapes and sizes the batch. The candy enters a former, which shapes the individual pieces into discs, balls, barrels, etc. The present invention can be made into any shape, circles, squares, triangles etc, also into animal shapes or any other novelty molding available. The candy is then cooled, wrapped and packaged.

For Grained Types of candy, water and sugar are the basic components being mixed with other ingredients, and cooked at high temperatures (290° F. 310° F.), causing the water to turn to steam. The product is transferred to a cooling wheel, where it is collected in about 150 pound batches, placed in a pulling machine to aerate the product, and the flavor is added. The candy is transferred to batch rollers where it is shaped and sized. The candy then enters a former, which shapes the individual pieces. The candy is cooled at a relative humidity of 35% and enters a rotating drum where it is coated with a fine sugar. The candy is then conveyed to the graining room for four hours at 90° F. and 60% humidity. The entrapped air and moisture causes the product to grain.

The present invention can be of a variety of shapes, flavors and sizes. The present invention may contain sugar or may be sugarless.

Flavors used in the present invention may be peppermint oils, citrus oils, arvensis, fruit flavors, spearmint oils and the like.

Colors used in the present invention are colorants are typically known as FD&C dyes and lakes.

By way of example and not limitation, the following examples illustrate various embodiments of the confectionery formulations of the present invention.

TABLE 6
Novel Flavor Candy Formulations (% by weight)

| Ingredient | Example 28 | Example 29 | Example 30 | Example 31 | Example 32 | Example 33 | Example 34 |
|---|---|---|---|---|---|---|---|
| Corn Syrup | 44.51 | 43.25 | — | — | 48.00 | — | — |
| Sugar | 53.49 | 50.00 | — | — | 46.97 | — | — |
| Polyalcohols | — | — | 95.20 | 95.77 | — | — | — |
| Erospicata | 0.75 | 1.92 | 2.80 | 0.48 | 3.15 | 0.50 | .25 |
| Color | 0.49 | 1.00 | 0.60 | 0.80 | 0.50 | — | — |
| Cooling Agent | 0.67 | 1.92 | 1.20 | 0.65 | 0.45 | 0 | — |
| High Intensity | — | — | 0.20 | 0.20 | — | — | — |
| Sweetener | | | | | | | |
| Peppermint | 0.08 | 1.91 | — | 2.10 | 0.90 | — | .50 |
| Heating Agent | 0.01 | — | — | — | 0.03 | — | — |
| Menthol | | | | | | 0.50 | .25 |
| Total % | 100.00 | 100.00 | 100.00 | 100.00 | 100.00 | | |

Example 33. Any of examples 1-32 is prepared with the cooling agent being 100% N-ethyl-p-menthane-3-carboxamide.

Example 34. Any of examples 1-32 is prepared with the cooling agent being N,2,3-trimethyl-2-isopropyl-butanamide.

Any of the examples in 1-32 may be prepared with the cooling agent ingredient being 100% of one of the non-menthol cooling agents disclosed, or is a blend of cooling agents as represented, by way of example, in the following table.

TABLE 7
Possible Blends of Cooling Agents

| Cooling Agent | Example A | Example B | Example C | Example D | Example E |
|---|---|---|---|---|---|
| WS-23 | — | — | — | 6 | 4 |
| WS-3 | — | 17 | — | — | 6 |
| Menthyl Glutarate | — | — | — | 23 | — |
| Menthyl Succinate | — | 25 | — | — | — |
| Menthol PG carbonate | 25 | — | 50 | — | — |
| Menthol EG carbonate | 25 | 19 | — | 10 | — |
| Menthyl lactate | — | 21 | — | 5 | — |
| Menthone glyceryl ketal | 25 | — | — | 14 | — |
| Menthol glyceryl ether | — | — | 50 | — | 90 |
| Homomenthol | 25 | — | — | 19 | — |
| Isopulegol | — | 18 | — | 23 | — |
| Total | 100 | 100 | 100 | 100 | 100 |

TABLE 8
Possible Blends of Cooling Agents

| Cooling Agent | Example F | Example G | Example H | Example I | Example J |
|---|---|---|---|---|---|
| WS-23 | — | — | — | 70 | — |
| WS-3 | — | — | 30 | — | — |
| Menthyl Glutarate | — | — | 20 | — | 5 |
| Menthyl Succinate | — | 6 | — | — | 18 |
| Menthol PG carbonate | 33 | 27 | 10 | — | — |
| Menthol EG carbonate | 33 | — | 10 | — | — |
| Menthyl lactate | 33 | 12 | — | — | 61 |
| Menthone glyceryl ketal | — | — | 30 | 10 | — |
| Menthol glyceryl ether | — | 39 | — | 10 | 16 |
| Homomenthol | 1 | — | — | 10 | — |
| Isopulegol | — | 16 | — | — | — |
| Total | 100 | 100 | 100 | 100 | 100 |

TABLE 9
Possible Blends of Cooling Agents

| Cooling Agent | Example K | Example L | Example M | Example M | Example O |
|---|---|---|---|---|---|
| WS-23 | 10 | 5 | — | 50 | — |
| WS-3 | — | 20 | — | 50 | — |
| Menthyl Glutarate | 20 | — | 10 | — | 5 |
| Menthyl Succinate | — | — | 10 | — | 20 |
| Menthol PG carbonate | 50 | — | 10 | — | — |
| Menthol EG carbonate | — | 30 | 10 | — | 10 |
| Menthyl lactate | — | — | 10 | — | 20 |
| Menthone glyceryl ketal | — | — | 10 | — | 7 |
| Menthol glyceryl ether | — | 45 | 20 | — | 13 |
| Homomenthol | 20 | — | 10 | — | — |
| Isopulegol | — | — | 10 | — | 25 |
| Total | 100 | 100 | 100 | 100 | 100 |

It should be understood that various changes and modifications to the presently preferred embodiments described herein will be apparent to those skilled in the art. Such changes and modifications can be made without departing from the spirit and scope of the present invention and without diminishing its intended advantages. It is therefore intended that such changes and modifications be covered by the appended claims.

The invention is claimed as follows:

1. A consumable oral product comprising Erospicata oil and an agent chosen from the group consisting of a cooling agent and a heating agent.

2. The consumable oral product of claim 1 wherein the cooling agent is selected from the group consisting of: N-ethyl-p-menthane-3-carboxamide, N,2,3-trimethyl-2-isopropyl-butanamide, menthyl glutarate, menthyl succinate, menthol PG carbonate, menthol EG carbonate, menthyl lactate, menthone glyceryl ketal, menthol glyceryl ether, 3,3,5-trimethylcyclohexanol, and isopulegol.

3. The consumable oral product of claim 1 wherein the heating agent is chosen from the group consisting of: capsicum oleoresin, capsaicin, piperine, gingerol, shoagol, cinnamic aldehyde, ginger oleoresin, cinnamon oleoresin, and cassia oleoresin, black pepper oleoresin, and pepper oleoresin

4. The consumable oral product of claim 1 wherein the agent is present in an amount of from about 0.01% to about 2.0% by weight.

5. The consumable oral product of claim 1 comprising both a heating agent and a cooling agent.

6. The consumable oral product of claim 1 wherein the Erospicata oil comprises approximately about 0.01% to about 5% by weight of the product.

7. The consumable oral product of claim 1 wherein the product is selected from the group consisting of chewing gum, dentifrice, confection, lozenge, mouthwash, mouth spray, and edible film.

8. The consumable oral product of claim 1 wherein the Erospicata oil is present as a flavoring agent.

9. The consumable oral product of claim 8 wherein the flavoring agent comprises 0.05 to 15% by weight of the product.

10. The consumable oral product of claim 8 wherein Erospicata oil comprises 5 to 70% of the flavoring agent.

11. A chewing gum product comprising Erospicata oil and a cooling agent.

12. The chewing gum product of claim 11 wherein the cooling agent is selected from the group consisting of: N-ethyl-p-menthane-3-carboxamide, N,2,3-trimethyl-2-isopropyl-butanamide, menthyl glutarate, menthyl succinate, menthol PG carbonate, menthol EG carbonate, menthyl lactate, menthone glyceryl ketal, menthol glyceryl ether, 3,3,5-trimethylcyclohexanol, and isopulegol.

13. The chewing gum product of claim 11 comprising a heating agent chosen from the group consisting of: capsicum oleoresin, capsaicin, piperine, gingerol, shoagol, cinnamic aldehyde, ginger oleoresin, cinnamon and cassia oleoresin, black pepper oleoresin, pepper oleoresin

14. The chewing gum product of claim 11 wherein the cooling agent is present in an amount of from about 0.01% to about 2.0% by weight.

15. The chewing gum product of claim 11 comprising a heating agent.

16. The chewing gum product of claim 11 wherein the Erospicata oil comprises approximately about 0.01% to about 5% by weight of the chewing gum product.

17. The chewing gum product of claim 11 wherein the cooling agent is selected from the group consisting of menthol, menthol glyceryl ether, N,2,3-trimethyl-2-isopropyl-butanamide and menthyl glutarate.

18. The chewing gum product of claim 11 wherein the Erospicata oil is present as a flavoring agent.

19. The chewing gum product of claim 18 wherein the flavoring agent comprises 0.05 to 15% by weight of the product.

20. The chewing gum product of claim 18 wherein Erospicata oil comprises 5 to 70% of the flavoring agent.

21. An edible film product comprising Erospicata oil and a cooling agent.

22. The edible film of claim 21 wherein the cooling agent is selected from the group consisting of: N-ethyl-p-menthane-3-carboxamide, N,2,3-trimethyl-2-isopropyl-butanamide, menthyl glutarate, menthyl succinate, menthol PG carbonate, menthol EG carbonate, menthyl lactate, menthone glyceryl ketal, menthol glyceryl ether, 3,3,5-trimethylcyclohexanol, and isopulegol.

23. The edible film of claim 21 comprising a heating agent chosen from the group consisting of: capsicum oleoresin, capsaicin, piperine, gingerol, shoagol, cinnamic aldehyde, ginger oleoresin, cinnamon and cassia oleoresin, black pepper oleoresin, pepper oleoresin.

24. The edible film of claim 21 wherein the cooling agent is present in an amount of from about 0.01% to about 2.0% by weight.

25. The edible film of claim 21 comprising a heating agent.

26. The edible film of claim 21 wherein the Erospicata oil comprises approximately about 0.01% to about 5% by weight of the product.

27. A method of enhancing a flavor in an oral consumable product comprising the steps of adding to a consumable oral product Erospicata oil and an agent chosen from the group consisting of a heating agent and a cooling agent.

28. The method of claim 27 wherein the oral consumable product is selected from the group consisting of chewing gum, dentifrice, confection, lozenge, mouthwash, mouth spray, and edible film.

29. The method of claim 27 wherein the Erospicata oil is present as a flavoring agent.

30. The method of claim 29 wherein the flavoring agent comprises 0.05 to 15% by weight of the product.

31. The method of claim 29 wherein Erospicata oil comprises 5 to 70% of the flavoring agent.

32. A method of producing a chewing gum product comprising the steps of adding to a composition that includes a gum base Erospicata oil and an agent chosen from the group consisting of a cooling agent and a heating agent.

33. A method of reducing the amount of peppermint flavor required to achieve an organoleptic effect in a product comprising the steps of manufacturing the product with Erospicata oil and a cooling agent.
